(19) United States
(12) Patent Application Publication
(10) Pub. No.: US 2011/0208161 A1
(43) Pub. Date: Aug. 25, 2011

(54) INTRACOCHLEAR DRUG DELIVERY TO THE CENTRAL NERVOUS SYSTEM

(76) Inventor: Yehuda Ivri, Newport Beach, CA (US)

(21) Appl. No.: 12/855,152

(22) Filed: Aug. 12, 2010

Related U.S. Application Data
(60) Provisional application No. 61/274,141, filed on Aug. 13, 2009.
(60) Provisional application No. 61/276,727, filed on Sep. 16, 2009.
(60) Provisional application No. 61/279,463, filed on Oct. 21, 2009.
(60) Provisional application No. 61/284,064, filed on Dec. 12, 2009.

(51) Int. Cl. A61F 11/00; A61M 31/00

(52) U.S. Cl. 604/514; 604/257

(57) ABSTRACT

The present invention is directed to method and system for delivery of brain-targeted drugs to the cerebrospinal fluid via the perilymphatic fluid of the inner ear. The system utilizes the passage of cochlear aqueduct as a drug delivery route from the inner ear to the subarachnoid space of the brain. The delivery system includes an otological conduit which enables transfer of drugs from the auditory ear canal to the inner ear and a wearable dispenser for supplying drugs to the otological conduit. The drug composition comprises a suspension of solid lipid nanoparticles (SLN) which facilitate delivery through the cochlear aqueduct. Employing aspects of present invention, a method and system for treating chronic pain is described.

This application claims benefit of Provisional Application Ser. No. 61/279,463 filed Oct. 21, 2009 and of Provisional Application Ser. No. 61/276,727 filed Sep. 16, 2009 and of Provisional Application Ser. No. 61/274,141 filled Aug. 13, 2009 and of Provisional Application 61/284,064 filled Dec. 12, 2009, which applications are hereby incorporated herein by reference in their respective entireties to the extent that they do not conflict with the present disclosure.

### FIELD OF INVENTION

The present invention relates to prolonged administration of brain-targeted therapeutics to the cerebrospinal fluid (CSF) via the perilymphatic fluid of the inner ear of a mammal or a human being.

### BACKGROUND OF THE INVENTION

Currently, there are a number of methods and devices available to deliver medications to cerebrospinal fluid (CSF) in the spinal canal or in the subarachnoid space of the brain. The majority of them are based on continuous infusion by means of catheters inserted into the spinal structure or through the scalp into the ventricular lumen of the brain. Mostly, the drugs for such delivery are opioid analgesics which are delivered directly to the CSF to avoid systemic exposure and related side effect. Presently, spinal analgesics are delivered by a pump which is surgically implanted under the skin in the abdominal space. The drug is then delivered to the cerebrospinal fluid via the intrathecal route. Alternatively drugs may be delivered by an intraventricular catheter system known as Ommaya reservoir. The catheter is inserted through a port in the scalp directly to the cerebrospinal fluid in a ventricular cavity of the brain. This method is mainly used to deliver chemotherapy to the CSF in treatment of brain tumor and to deliver analgesics opioid in treatment of intractable pain.

The current method for intrathecal treatment of chronic pain is by means of an intrathecal pump, such as the SynchroMed® Infusion System, a programmable, implanted pump available from Medtronic Inc., of Minneapolis, Minn. The system includes a catheter and a pump section. The system automatically delivers a controlled drug amount through the catheter to the cerebrospinal fluid (CSF) by means of an electric peristaltic pump. At present, the SynchroMed® is used for spinal delivery of antinociceptive or antispasmodic therapeutics and has been proposed for delivery of large molecule such as peptides and hormones to the CSF in a ventricular lumen of the brain.

Because of the short half-life of these substances they require frequent re-administration, and this is realized by the implanted pump. Although, the system has some major advantages over other existing methods, it also has some disadvantages. One disadvantage is the large, bulky size of the SynchroMed® pump. Due to its size, the device must typically be implanted in the abdominal cavity of a patient and an extended catheter has to be passed through the patient's body to deliver the drug to the desired site of administration. In addition to problems with size and placement, the SynchroMed® is burdened by complex electronics for both programming and pumping functionality. Furthermore, complications may arise as a result of the required surgical implantation and the possibility of leakage of the catheter as well as of the pump.

As a result from these limitations the numbers of patients who are qualified or choose to use intrathecal pump treatment is limited. There is therefore a need for less invasive methods of delivery of therapeutic molecules to the CSF with a reduced risk associated with the invasiveness of intrathecal or intracerebroventricular drug delivery.

An animal study has shown that the inner ear can be used as a drug delivery route to the cerebrospinal fluid. The perilymphatic fluids in the compartment of the inner ear are connected to the cerebrospinal fluid in the subarachnoid space of the brain via the passage of the cochlear aqueduct. It has been shown that in a live animal, there exists a physiological flow from the compartment of the inner ear to the CSF in the subarachnoid space of the brain. A study that investigated the potential use of the inner ear as a delivery route to the CSF has shown that drugs effectively pass from the round-window membrane (RWM) of the inner ear to the CSF following a single trans-tympanic injection.

The present invention provides a minimally invasive drug delivery means for prolonged administration to the CSF via the round-window membrane of the inner ear. The methods and systems described herein allow for improved surgical ease relative to intracerebroventricular (ICV) or intrathecal method.

### BRIEF SUMMARY OF THE INVENTION

The present invention is directed to methods and systems for drug delivery to the cerebrospinal fluid (CSF) via inner ear. More specifically, the invention directed to a minimally invasive method for prolonged delivery of brain-targeted therapeutics to the CSF via the perilymphatic fluid of the inner ear.

The perilymphatic fluid inside the inner ear and the cerebrospinal fluid (CSF) surrounding the central nervous system (CNS) are fluidly connected to one another via the passage of the cochlear aqueduct. Animal studies have shown that the perilymphatic fluid physiologically flows from the inner ear to the CSF in the subarachnoid compartment of the brain. An animal study that investigated the feasibly of using the inner ear as a delivery route to the CSF has shown that dexamethason acetate loaded to solid-lipid nanoparticles (SLN) effectively transports to the CSF following trans-tympanic injection.

While dexamethason acetate has long half-life in the CSF, most brain-targeted therapeutics requires frequent re-administration which is impractical to realize by trans-tympanic injection. The present invention provides a minimally invasive drug delivery system and method which overcomes this limitation. The delivery system is configured for prolonged administration of brain-targeted therapeutics to the CSF via the auditory ear canal. The system may be used for treatment of chronic CNS diseases and disorders, including pain.

The delivery system is used for prolonged administration of fluid composition to the perilymphatic fluid of the inner ear. The system comprises of an implantable otological conduit which extends from the auditory ear canal to the round-window membrane, the conduit receives fluid from a nozzle in the auditory ear canal and deliver said fluid to the round-window membrane. The delivery system further comprising a dispenser which includes a dispensing nozzle connected to a fluid cartridge and an electronic module, the dispensing nozzle is placed inside the auditory canal in a closed proximity to the otological conduit. The dispensing nozzle operates intermittently to supply said fluid composition to the otological conduit based on a pre-programmed setting.

Employing aspects of the delivery system described herein, a method for treating chronic pain is described. The method comprising filling fluid analgesics into a cartridge which is connected to a dispensing nozzle. The analgesics are selected from a group consisting of analgesics opioid or α2-adrenergic agonist or a combination thereof. The method further includes implanting an otological conduit in the tympanic membrane for delivery of fluid from the auditory ear canal to the round window membrane, placing said dispensing nozzle inside the ear canal near the otological conduit and dispensing the fluid composition from the cartridge to the otological conduit at a predetermined delivery rate, such that the fluid composition perfuse through the round-window membrane to the perilymphatic fluid.

Conveniently, the dispensing device of the present invention may be worn on the external ear as a removable hearing aid product, on any part of the body or on a clothing article. The dispensing device operates intermittently to supply fluid composition to the otological conduit inside the ear canal. The otological conduit conveys fluid to the site of the round-window membrane. Subsequently, fluid passes through the RWM by perfusion to the perilymphatic fluid inside the inner ear. The physiological flow of the perilymphatic fluid to the cerebrospinal fluid (CSF) carries the drug to subarachnoid space of brain and the dynamic flow within the subarachnoid space distributes the drug in the brain's compartments.

The delivery systems described herein, allow for improved surgical ease relative to others drug delivery systems to the CSF such as intracerebroventricular (ICV), intraspinal and intrathecal.

### BRIEF DESCRIPTION OF THE DRAWINGS

FIG. 1 is a perspective view of the dispensing device in accordance to example embodiment of the invention.

FIG. 2 is a perspective exploded view of the dispensing device of FIG. 1.

FIG. 3 is a cross sectional view of the thermoelectric dispensing nozzle of the dispenser of FIG. 1.

FIG. 4 is a cross sectional view of the tube member of the dispensing device in FIG. 1.

FIG. 5 is the dispensing device of FIG. 1 shown in placement behind a human ear.

FIG. 6 is the dispensing device of FIG. 1 shown inside a human ear.

FIG. 7 is an enlarged view of the otological conduit shown in FIG. 6.

FIG. 8 is the dispensing device of FIG. 1 in a placement behind a human ear.

FIG. 9 is the dispensing device of FIG. 1 with an alternative otological conduit shown inside a human ear.

FIG. 10 is an enlarged view of the otological conduit of FIG. 9.

FIG. 11 is a dispensing device in accordance to example embodiment of the invention for placement inside the ear.

FIG. 12 is a dispensing device in accordance to example embodiment of the invention for configured for placement as wearable earphone.

FIG. 13 is the dispensing device of FIG. 12 shown in placement on a human ear.

FIG. 14 is a schematic diagram showing typical flow patterns of cerebrospinal fluid through a human central nervous system.

### DETAILED DESCRIPTION OF THE INVENTION

In the following detailed description, reference is made to the accompanying drawings that form a part hereof, and in which are shown by way of illustration several specific embodiments of devices, systems and methods for prolong delivery of brain-targeted therapeutics to the CSF via the perilymphatic fluid of the inner ear. The method utilizes the physiological flow between the inner ear and the CSF as well as the dynamic distribution of the CSF in the brain compartment to facilitate drug delivery to the CSF. It is to be understood that other embodiments which deliver drug to the perilymphatic fluid are contemplated and may be made without departing from the scope or spirit of the present invention. The following detailed description, therefore, is not to be taken in a limiting sense.

Devices and methods for delivering molecules to the central nervous system (CNS) are discussed. The devices and methods described allow for less invasive procedures to be employed for delivering molecules to the brain.

### DEFINITIONS

All scientific and technical terms used herein have meanings commonly used in the art unless otherwise specified. The definitions provided herein are to facilitate understanding of certain terms used frequently herein and are not meant to limit the scope of the present disclosure.

As used herein, “subject” means an animal which utilized the intracochlear delivery system and may be and includes mammals, such as humans.

As used herein, “treatment” or “treating” means to subjectively or objectively alleviate at least one symptom of a disease.

As used herein, “disease” means a condition of a subject or a portion thereof that impairs normal functioning and is typically manifested by signs or symptoms, condition, disease, disorder and the like are used herein interchangeably. Sign and symptom are used herein interchangeably. Sign and symptom are used herein interchangeably.

As used herein, “large molecule” means a molecule having a peptide bond, such as a polypeptide, or a molecule having a phosphodiester bond, such as a polynucleic acid.

As used herein, “polypeptide” means a molecule comprising an amino acid or a derivative thereof joined by a peptide bond to another amino acid or derivative thereof and typically refers to a protein having an activity on a biological system. It will be understood that referral to a specific polypeptide, such as leptin, includes any polypeptide having activity substantially similar to the specific polypeptide. “Polypeptide” and “peptide” are used herein interchangeably.

Delivery System:

Any wearable dispensing system having a reservoir from which fluid is supplied to the auditory ear canal and transferred by an otological conduit to the round-window membrane of the inner ear may be employed in accordance with the methods described herein.

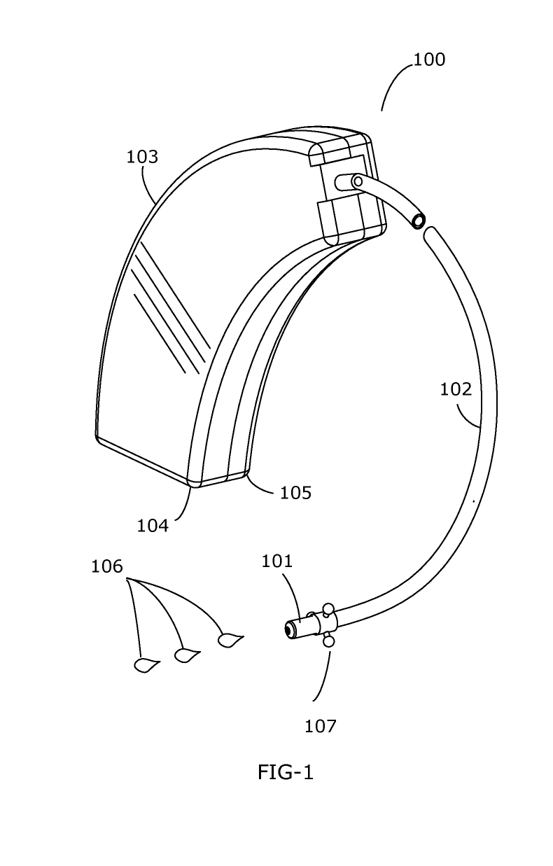

Referring now to FIG. 1, a representative dispensing system accordance to example embodiment of the invention is shown. Dispenser 100 is designed to be positioned behind the ear as hearing aid products commercially known as BET (Behind-the-Ear). Dispenser 100 includes a thermoelectric dispensing nozzle 101 connected by a tube member 102 to a housing 103 which contains a sealed fluid cartridge 104 and an electronic control module 105. Tube member 102 is formed in a preformed shape and is made of materials with are sufficiently rigid to support the dispensing nozzle in a proper position inside the subject's ear canal. Tube member 102 further includes an ear-tip member 107 for anchoring the end of the tube and the nozzle 101 within the ear canal of a subject. Fluid, e.g. fluid containing brain-targeted therapeutic agent may be stored in the cartridge 104. Dispensing device 100 operates to dispense fluid 106 based on preprogrammed setting of the electronic control module 105.

In one example embodiment the dispensing nozzle 101 is a thermoelectric dispensing nozzle which propels fluid by expansion of bubbles produces by thermal energy.

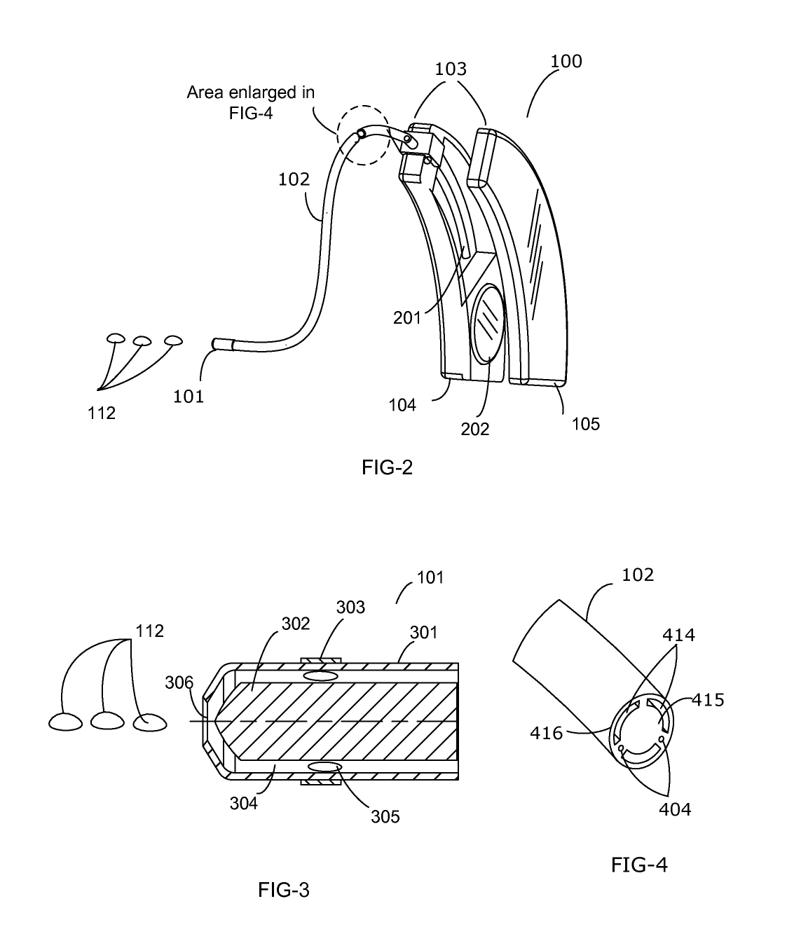

Referring to FIG. 2, an exploded view of housing 103 is shown. Housing 103 comprises a disposable drug cartridge 104, a reusable electronic module 105, and an electrical interface between the two. The disposable cartridge 104 comprises a sealed chamber 201 containing a supply of liquid to be dispensed and a coin cell battery 202. The electronic module 105 comprises an electronic circuit that controls operation of the dispenser. The interface between the drug cartridge module 104 and the electronic module 105 attaches the two modules mechanically, makes an electrical connection between the coin cell battery 202 and the electronic control circuit, and makes an electrical connection between the electronic control circuit and the thermoelectric nozzle. Battery 202 is a 3-volt coin cell type CR2012 selected due to its compactness which enables convenient packaging and placement of the housing 103 behind the ear.

Referring to FIG. 3, a cross-sectional view of the thermoelectric nozzle 101 is shown. Thermoelectric nozzle 101 comprises of a thin outer wall member 301, a core member 302 and a thermoelectric resistive layer 303. The thin outer walled member 301 and the core 302 are integrally connected in an axis-symmetric relationship by a rib member (not shown). The diameter of the core member 302 is slightly smaller than the inside diameter of the thin outer wall member 301 such that an annular gap 304 is formed between the two members. The radial gap 304 defines an annular capillary channel through which fluid flows. The electrical resistive layer 303 is deposed on the surface 301 of the outer wall. Passage of electrical current through the resistive layer 303 generates heat, sometime called joule or ohmic heating. Heat from the resistive layer conducts through the thin outer wall 301 and causes near instantaneous boiling of liquid in the annular gap 304. The boiling may be nucleus boiling. Nucleus boiling refers to a rapid boiling process that changes the state of a liquid to a gas exclusively at a selected region while the liquid remains in liquid form in other nearby regions. As some of the liquid is vaporized, one or more bubbles 305 form in the annular gap 304. Expansion of the one or more bubbles forces a quantity of liquid 112 out of opening 306 of the nozzle 101.

The thin outer wall member 304 is made from materials that have low thermal conductivity to prevents distribution of heat energy in the nozzle and to facilitates local heating and nuclear boiling exclusively in the vicinity of the resistive layer 303. The outer wall is made of borosilicate glass or glass silica which has thermal conductivity of about 1.2 W/m K (watt/meter deg. Kelvin). Other types of glass or ceramics which have thermal conductive below 5 W/m K may be used.

The resistive layer 303 is made from materials which have specific electrical resistance (resistivity) in the range of 200×10⁸ Ωm to 1000×10⁸ Ωm. Resistive layer may be deposit by a sputtering process or by screen printing using a paste-like material following by a firing process. Such printing process is used in fabrication of microelectronic circuit (see, e.g., U.S. Pat. No. 5,256,836).

The thermoelectric dispensing technology allows miniaturization of the dispensing nozzle and the energy source. The technology is described in details in U.S. Pat. No. 7,673,820 (to Ivri) and is incorporated here by reference to the extent that it does not conflict with the disclosure presented herein. Miniaturization enables convenient placement of the dispenser 100 similarly to hearing aid products, for example, Behind-the-Ear (BET) and Inside-the-Ear (ITE) type hearing aid products.

Due to limited power output, coin cells batteries, such as CR2012, may not be able to supply energy sufficiently fast to form a bubble and dispense droplets. Specifically, the thermoelectric transducer of the present invention may require a power of about 0.3-2 watts. The power available from a miniature coin cell battery is typically about 0.030 watts, an order of magnitude smaller than the thermoelectric transducer requirement. To overcome this power limitation the circuit performs a two-step process to produce the power requirement. The first step is using the battery to charge a supercapacitor at a relatively slow rate and the second step is discharging the supercapacitor relatively rapidly to the thermoelectric transducer. The circuit may charge the supercapacitor over a period of several minutes or longer. The discharge through the thermoelectric transducer may be nearly instantaneous, taking place in the span of less than a second. The advantage of supercapacitor is that it reduces the size of the energy source in wearable dispensers that operate intermittently (“on” and “off”), wherein the “off” periods, or a part thereof, can be used to charge a supercapacitor. Supercapacitor may also be use with micro-fluidic dispensers which operate intermittently, such as dispensers that utilize piezoelectric or electromagnetic transducers.

The coin cell battery type CR2012 battery available from Energizer Holdings, Inc., of St Louis, Mo., USA has a maximum recommended current drain of 0.1 ampere. The circuit uses energy from battery to slowly charge a supercapacitor without exceeding the recommended current drain provided by the battery manufacturer. Supercapacitor may be, for example, an electric double layer super-capacitor model HA-130 manufactured by CAP-XX Ltd., of Lane Cove Australia. The model HA-130 supercapacitor is packaged in a flat form and has a thickness of about 1.5 mm and an energy density of 2 watt-hour/kg. This size, weight and energy density configuration is particularly suitable for compact packaging in housing behind the ear. The HA-130 supercapacitor is capable of producing an instantaneous pulse of up to 5 amperes at a voltage rating of about 2.3 volts. The preferred capacitance of the supercapacitor is about 0.9 Farad and the preferred voltage limit is about 2.7 volts. Other capacitors may also be used, preferably those with energy density in the range from 1-10 watt-hour/kg.

Referring to FIG. 4, an enlarged cross sectional view of the tube member 102 is shown. Tube member 102 is an annular capillary tube that supplies fluid from the cartridge to the nozzle by capillary action. The annular capillary passage is formed in the gap 414 between a central core member 415 and the outer wall 416. The gap is appropriately sized to draw liquid from the drug cartridge by capillary action. A gap size of 20 to 200 micron can generate capillary pressure which overcomes gravity. Preferably, the tube 102 is made of hydrophilic materials such as hydroxypropyl methylcellulose (HPMC-15), polymethyl methacrylate (PMMA), or poly hydroxyethyl methacrylate (pHEMA). In the preferred embodiment the external diameter of the tube is about 1.5 mm; however size range of 0.75 mm to 2.0 mm may be used. The tube is provided with 2 channels 404 through which electrical leads are conducting power to the dispensing nozzle.

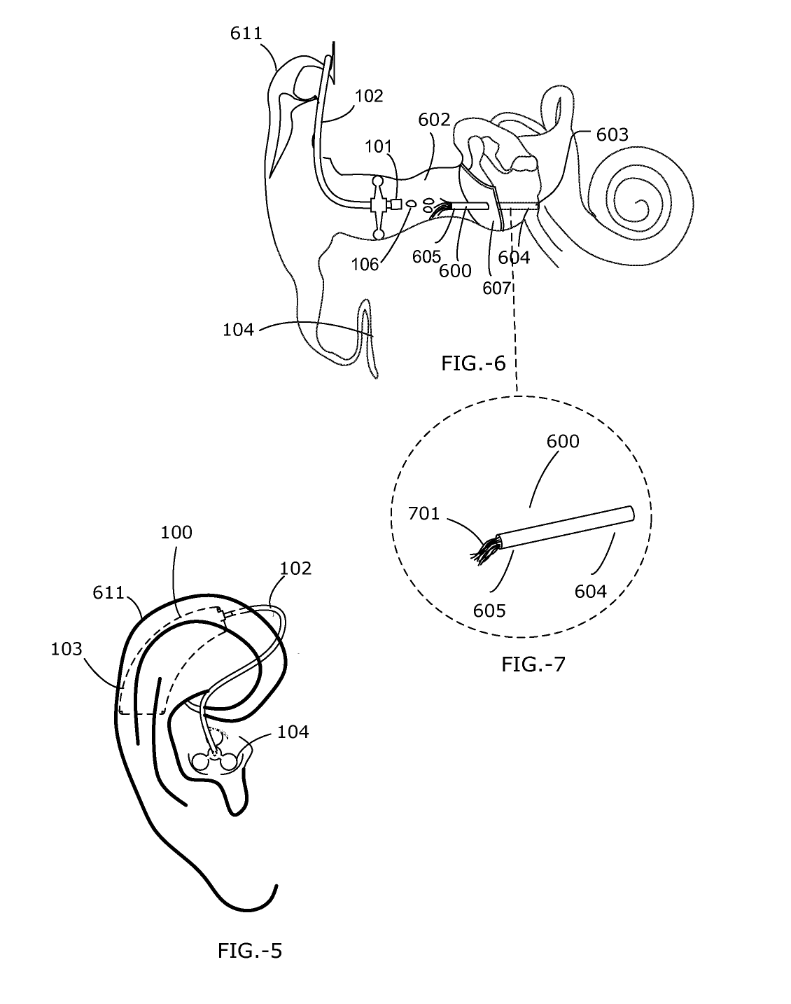

Referring to FIGS. 5 and 6, a representative delivery system in accordance with an example embodiment the present invention is shown within a side view and a cross sectional view of a human ear.

The delivery system comprises an otological conduit 600 for conveying fluid from the auditory ear canal 602 to the round-window membrane (RWM) 603 of the inner ear and a dispenser 100 for supplying fluid 106 to the otological conduit 600.

Otological conduit 600 is implanted in the tympanic membrane 607 and configured to deliver fluid from the ear canal 602 to the round-window membrane (RWM) 603. Conduit 600 comprising a wick member having a distal end 604 for contacting the RWM 603 and a proximal end 605 for contacting a fluid supply source inside the ear canal 602. The otological conduit is configured and dimensioned to pass through a tympanostomy tube which is implanted in the tympanic membrane 607. The otological conduit 600 is made of materials capable of conveying medication from the proximal end 605 to the distal end 604 by capillary action.

The otological implant 600 and the procedure for its placement in the tympanic membrane are described in detail in U.S. Pat. No. 6,120,484 (to Silvestein) which is hereby incorporated herein by reference to the extent that it does not conflict with the disclosure presented herein.

Dispenser 100 provides a source of fluid to the otological conduit 600. Dispenser 100 includes a thermoelectric dispensing nozzle 101 connected by a tube member 102 to a housing 103 which contains a hermetically sealed fluid cartridge and an electronic control module (shown in dashed lines to indicates that the housing 103 is hidden behind the ear). Dispensing nozzle 101 is positioned inside the auditory ear canal 602 in closed proximity, about 5 mm or less, from proximal end of the otological conduit 605 such that the fluid droplets 106 irrigate to the otological conduit 600. Fluid, such as drug composition, conveys through the otological conduit to the round-window membrane and enters the inner ear by perfusion due to concentration gradient between the drug composition and the perilymphatic fluid. Perfusion rate gradually slows as the concentration gradient equalized. Dispensing nozzle operates intermittently to replenish the drug composition such that a concentration gradient is maintained. In the delivery system of the present invention effective perfusion via the round-window membrane is achieved when the dispenser 100 delivers sufficient fluid to fully saturate the otological conduit 600 with fluid.

In one example embodiment the otological conduit 600 may be the Silverstein MicroWick™ Drug Delivery System, an implant that is clinically used for treatment of inner ear diseases. MicroWick™ Drug Delivery System and tympanostomy tubes of various styles are available from by Micromedics Inc. St Paul, Minn.

The physician places the tympanostomy tube in a small incision in the tympanic membrane then inserts the MicroWick™ through the tympanostomy tube and directs it to the round-window membrane. The Silverstein MicroWick™ inserts easily when it is dry and locks securely in place, due to expansion, after contact with fluid medicament.

In one example embodiment of the present invention the otological conduit is modified to enhance absorption of fluid medication from the dispenser 100.

Referring to FIG. 7, a perspective view of the modified otological conduit 300 is shown. The otological conduit 600 comprises an elongated wick member having a proximal end 605 and a distal end 604. The proximal end 605 is provided with plurality of absorbing fibers 701 which assist in collecting fluid from the floor of the ear canal and transfer fluid to the proximal end 605 of the otological conduit 600. The fibers extend from the proximal end 605 to a distance of about 3-10 mm. The absorbing fibers comprising a bundle of about 400 individual fibers which are made of Nylon type 11. The diameter of each fiber is about 5-50 micron. The diameter of the conduit 600 is about 1.0 mm and its length is about 9 mm. The conduit 300 is made of porous polyvinyl acetate (PVA). Due to porosity, conduit 600 conveys fluid from the proximal end to the distal end by capillary action.

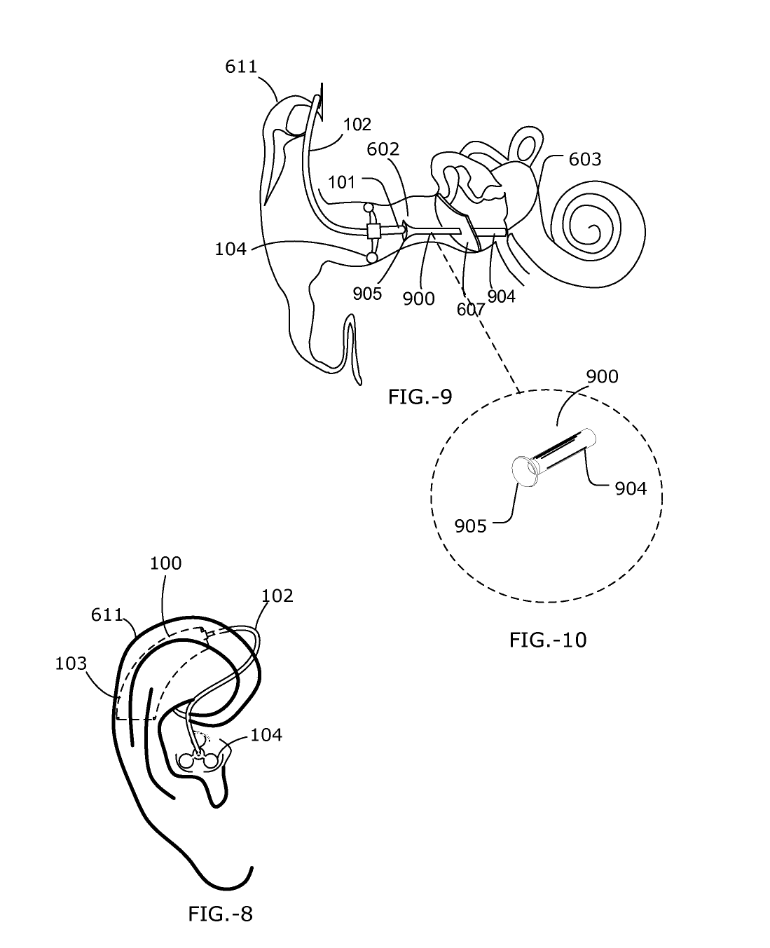

Referring to FIGS. 8 and 9 a cross sectional view and a side view of an alternative delivery system in accordance to example embodiment of the invention are shown inside a human ear.

The delivery system comprises an otological conduit 900 for conveying fluid from the auditory ear canal to the round-window membrane (RWM) of the inner ear and a dispenser 100 for supplying fluid to the otological conduit 900. An otological conduit 900 is implanted in the tympanic membrane and configured to convey fluid medicament from the ear canal 602 to the round window membrane (RWM) 603. The otological conduit 900 comprises a thin walled tube member having a distal end 904 for contacting the RWM 603 and a flared-mouth proximal end 905 for receiving a fluid medicament. Dispenser 100 provides a source of fluid to the otological conduit 900. Dispenser 100 includes a thermoelectric dispensing nozzle 101 connected by a tube 102 to a housing 103 which contains sealed fluid cartridge and an electronic control module (shown in dashed lines to indicates that the housing 103 is behind the ear). Dispensing nozzle 101 is positioned inside the auditory ear canal 602 in closed proximity, or in contact, with the flared mouth opening 905 of the otological conduit 900. Fluid is propelled from nozzle 101 passes through the tubular conduit 900 to the round-window membrane.

FIG. 10 shows an enlarged prospective view of the flared-mouth otological conduit 900. The outside diameter of the conduit is about 1.2 mm and inside diameter is about 0.8 mm. Other diameters may be used, preferably the outside diameter of the tube is 1-1.5 mm and the inside is about 0.5-1 mm. The external diameter of the flared-mouth opening 905 is about 4 mm. Other flared-mouth opening may be used preferably having an outside diameter between 2.5-5 mm The conduit is made of silicon rubber having a module of elasticity of 0.345 GPa to 2 GPa.

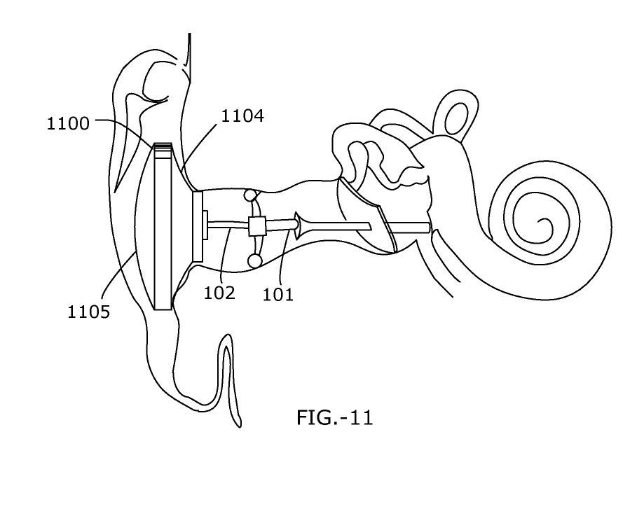

FIG. 11 shows an alternative dispensing in accordance to an example embodiment of the present invention. Dispenser 1100 is configured to be worn inside the ear as in inside-the-ear (ITE) hearing aid product. Dispenser 1100 includes a thermoelectric dispensing nozzle 101 connected by a tube member 102 to a housing 103 which contains a hermetically sealed fluid cartridge 1104 and an electronic control module 1205.

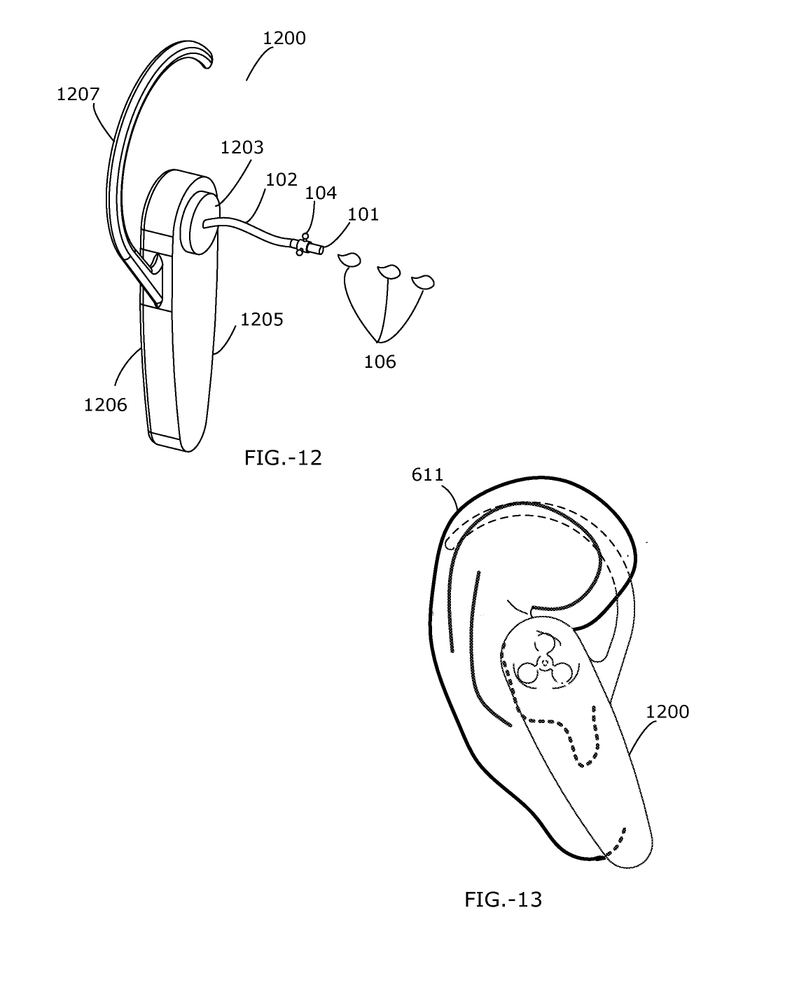

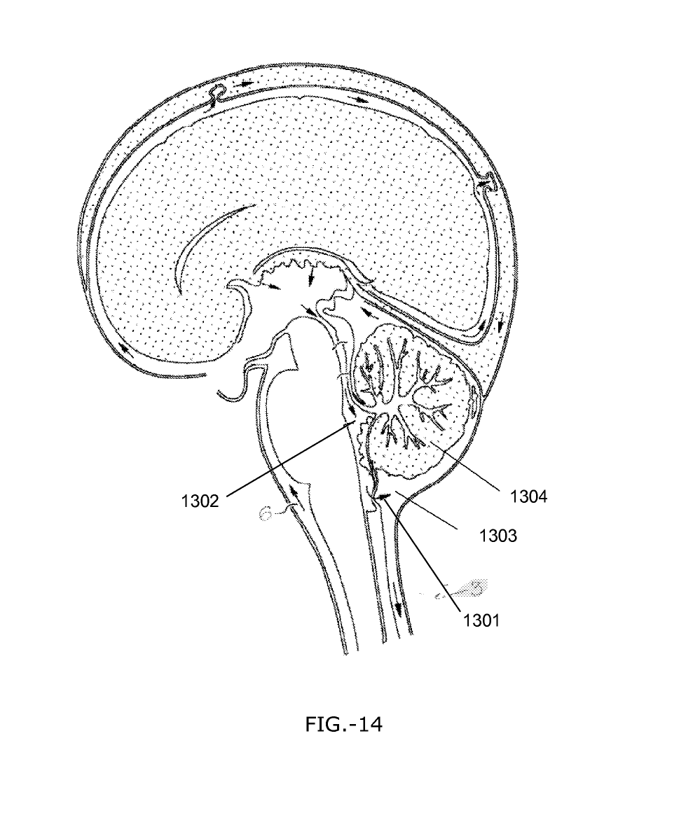

FIGS. 12 and 13 show an alternative dispensing device in accordance to example embodiment of the invention. Dispenser 1200 is configured to be worn outside the ear as earphone products commercially sold as Bluetooth earphone products. Dispenser 1200 includes a thermoelectric dispensing nozzle 101 connected by a tube member 102 to a housing 1205 which contains a hermetically sealed fluid cartridge 1203 and an electronic control module 1206. The dispensing device further includes ear-hook 1207. FIG. 13 shows the device worn on the ear 611 (dashed lines indicate that the ear-hook 1207 is behind the ear).

Delivery Via Inner Ear

The methods described herein relate to delivery of therapeutic compositions through the round-window membrane to the cerebrospinal fluid (CSF) of a subject, particularly to subarachnoid space via the perilymphatic fluid of the inner ear. Others delivery methods which employ catheters and/or cannulas to transport or recalculate fluids from a reservoir directly to the perilymphatic fluid may also be use for treatments described in the present invention. (see, e.g., Publication No. US 2006/0030837 A1)

The compartment of the inner ear is connected to the subarachnoid space of the brain via the passage of the cochlear aqueduct. Animal studies have shown the existence of physiological flow of perilymphatic fluid from the inner ear to the CSF (see, Kaupp H, et al, Distribution of marked perilymph to the subarachnoid space, Arch Otorhinolaryngol, 229(3-4): 245 [1980]). This flow is believed to provide the clearance mechanism which prevents accumulation of harmful products of metabolism inside the compartment of the inner ear. The study has shown that rhodamine dye that was applied to the round-window membrane (RWM) is transferred by perfusion to the inner ear and carried by the perilymphatic fluid to the CSF in the subarachnoid space of the brain. The present invention utilizes this flow to deliver drugs from the fluids of inner ear to the cerebrospinal fluid (CSF).

Delivery of drugs to the CSF via the inner ear was investigated in an animal study (see, Chen G. et al., Preliminary study on brain-targeted drug delivery via inner ear, Yao Xue Xue Bao, 42(10):1102 [2007]). The study provided the time-concentration curve in the CSF following administration of a free dexamethason solution or a suspension of solid-lipid nanoparticles (SLN). The study has shown a 13-fold increase in total dose in the CSF following delivery of dexamethason-loaded SLN. This may indicate that solid-lipid nanoparticles, a nano-colloidal carrier, enhances drugs delivery from the inner ear to the CSF via the narrow passage of the cochlear aqueduct. The cochlear aqueduct is often filled with meshwork of loose tissue which resists movement of fluids. The surface active layer reagents (lecithin, poloxamer) in solid-lipid nanoparticles is known to facilitate transport via tight restrictions such as the cochlear aqueduct.

Nano-colloidal carriers, such as solid-lipid nanoparticles may also be used to carry drugs that do not effectively pass through the cochlear aqueduct as a free molecule due to their physical properties such as lipophilicity, solubility or molecular weight.

Nano-colloidal carriers entrap drugs via encapsulation such that the physical properties of the drug molecule are not expressed until the encapsulation materials are eliminated by degradation. This degradation period allows sufficient time for the nanoparticles to cross the RWM and the cochlear aqueduct.

Further enhancement of intracochlear transport may be done by modification of the surface properties of nano-colloidal carriers. (see, Gabizon et al., Pharmacokinetics of pegylated liposomal doxorubicin, Clin Pharmacokinet 42: 419-436 [2003]). In drug delivery via inner ear, due to slow perfusion rate through the RWM, it is preferred to maximize the drug loading capacity of solid lipid nanoparticles. Lipophilic drugs, with good compatibility with the lipids, may be selected to incorporate into the SLNs to maximize drug loading and entrapment efficiency (see, Miglietta, et al, Cellular uptake and cytotoxicity of Solid Lipid Nanospheres (SLN) incorporating doxorubicin or paclitaxel. Int. J. Pharm., 210) and (Westesen et al, Physicochemical characterization of lipidnanoparticles and evaluation of their drug loading capacity and sustained release potential. J. Control Release, 48: [1997]). Generally, the prerequisite to obtain a sufficient loading capacity is further described in several references (see, e.g., Muller et al, Solid Lipid Nanoparticles (SLN) for controlled drug delivery—a review of the state of the art. Eur. J. Pharm. Biopharm., 50: 161-177. [2000]). Solid lipid nanoparticles may be formulated to maximize drug loading and entrapment efficiency according to techniques referenced herein or in any technique that is known in the art.

Method to prepare SLNs, include: high pressure homogenization, solvent emulsification or evaporation, high speed stirring ultrasonication and solvent diffusion method.

Nanoparticles may be composed of biodegradable polymers such as poly(alkylcyanoacrylates), polyesters such as poly(lactic acid), poly(glycolic acid), poly(_-caprolactone) and their copolymers, poly(methylidene malonate), and polysaccharides. Poly(lactic-co-glycolicacid) (PLGA) is one of the most common polymers used in making nanoparticles, because of its safety, biocompatibility, and long use in delivery systems and devices approved by the U.S. Food and Drug Administration.

The delivery system of the present invention may utilize nano-colloidal carriers to deliver pharmaceutical products or biological products such as recombinant therapeutic protein, vaccines, gene-based or gene transfer materials, cellular biologics, immunological medicinal product and cell therapy materials.

Nanocolloidal carriers include:
1. Polymeric nanoparticles (NPs) which are made of biodegradable polymers, and entrap drugs via encapsulation or polymer-drug conjugation
2. Liposomes which are made up of phospholipids and contain an aqueous core surrounded by a lipid bilayer. Hydrophilic drugs can be incorporated into the core; hydrophobic and amphiphilic drugs can be integrated into the bilayer.
3. Polymeric micelles which are formed in an aqueous environment from the associations of block copolymers containing both hydrophilic and hydrophobic segments. The hydrophobic core can be loaded with lipophilic drugs, and the hydrophilic surface serves to increase the stability of the micelles in water.
4. Dendrimers which contain many polymeric monomers that form branched, tree-like structures, allowing drugs to be attached to its many arms.
5. Carbon nanotubes which are made of benzene rings, can carry drugs inside the lumen of the tube or attached to the sides.

Treatment of Pain

In treatment of intractable cancer pain, general practice has been to treat patients with oral, parenteral or spinal morphine. However, due to the large dosage required by these patients, pain cannot be controlled without unacceptable side effects. To overcome this problem morphine and other opioid analgesics have been administered directly to the cerebrospinal fluid by a catheter through the scalp and into the ventricular compartment of the brain (see, e.g., Intracerebroventricular administration of morphine for control of irreducible cancer pain, Neurosurgery, 37(3):422-8 [1995]). This and other areas of the brain have high density of pain receptors, therefore, the treatment produces excellent pain relief with a small dose of morphine (see, Sandouk et al., Morphine pharmacokinetics and pain assessment after intracerebroventricular administration in patients with terminal cancer, Clin Pharmacol Ther., 49:442 [1991]). The study has shown that administration of morphine hydrochloride to the lateral ventricle at a dose of 0.01 mg/kg generally provide analgesic effect for about 24 hours in patient with severe intractable cancer pain.

Referring back to the study on brain-targeted drug delivery via inner ear. (see, Chen G. et al., Preliminary study on brain-targeted drug delivery via inner ear, Yao Xue Xue Bao, 42(10):1102 [2007]). Importantly, samples for the pharmacokinetic study described therein were extracted from the fourth ventricle of the brain. This indicates that drugs delivered via the inner ear are distributed in the brain's fourth ventricle and suggests that the delivery via the inner ear may be used in targeting the tissue of the brain inside and at the vicinity of the fourth ventricle. One such therapeutic application is in treatment of pain. The floor of the fourth ventricle has been implicated in the control of nociceptive transmission at the spinal level. Infusion of morphiceptin, a highly selective μ receptor agonist into the fourth ventricle, attenuate spinal cord nociceptive transmission (see, M D Mauk et al., Opiates and classical conditioning: Selective abolition of conditioned responses by activation of opiate receptors within the central nervous system, Psychology, 79: 7598 [1982]).

Additionally, the floor of the forth ventricle has high density of α2-adrenergic receptors. Delivery of α2 adrenergic receptor agonist, such as clonidine to the forth ventricle has been used in treatment of naturopathic pain or to ease withdrawal symptoms associated with the long-term use of narcotics, alcohol and nicotine, to treat migraine headaches, hot flashes associated with menopause, and in treatment of attention deficit hyperactivity disorder.

In treatment of naturopathic pain clonidine has been delivery directly to the CSF using implanted intrathecal pump. Clonidine is the most studied and the only FDA-approved α2-adrenergic agonist for intrathecal use. Intrathecal clonidine has been reported to provide significant analgesia, alone or in combination with opioid, for neuropathic pain, cancer pain, or complex regional pain syndrome. Direct delivery to the CSF prevents peripheral α2-adrenergic agonist side effects which may lead to hypertension.

Clonidine-hydromorphone mixtures has been delivery by implantable intrathecal pump for long-term use (see, Rudich et al., Stability of clonidine-hydromorphone mixture from implanted intrathecal infusion pumps in chronic pain patients, J Pain Symptom Manage 2004; 28:599).

Delivery of opioid analgesics or α-adrenergic receptor agonist via inner ear may therefore be used for treatment of pain; however, in contrast to dexamethason which has long half-life in the CSF, opioid analgesics are eliminated rapidly from the CSF due to physiological activities. Opioid analgesics, such as morphine, require frequent re-administration which is impractical to realize by trans-tympanic injections. The present invention overcomes this problem by providing a minimally invasive delivery system for prolonged administration to the CSF via the inner ear.

The delivery system includes an otological conduit implanted in the tympanic membrane and configured to convey fluid from the auditory ear canal to the round-window membrane, the system further includes a dispensing device which includes a dispensing nozzle positioned inside the auditory ear canal and supplies fluid analgesic to the otological conduit based on a preprogrammed setting. The dispensing device contains an electronic control and a drug cartridge which are connected to the dispensing nozzle by a tube member.

Efficiency of drug delivery from the inner ear to the CSF depends on the patency of the cochlear aqueduct. The patency varies between subjects and among age group (see, A J Phillips et al., Effects of posture and age on tympanic membrane displacement measurements, British Journal of Audiology, 23(4): 279 [1989]). When treating chronic pain it may be desirable to adjust the drug concentration, based on the patency of the cochlear aqueduct. For example a drug with higher concentration may be prescribed to a subject with a restricted cochlear aqueduct. Further adjustments in the delivery rate may be done based on the subject's pain relief. Opioid which have higher potency than morphine may be selected to overcome low patency of the cochlear aqueduct. For example hydromorphone hydrochloride which is 8-10 time more potent than morphine may be selected. Other opioid analgesics with higher potency may also be used, for example, alfentanil, fentanyl, fentanyl citrate, remifentanil, buprenorphine hyrochloride, or sufentanil. Opioid analgesics or α2-adrenergic agonist may be formulated as a free solution, a suspension of solid-lipid nanoparticles or as any other class of nanocolloidal carriers.

Delivery rate may also be adjusted according to dosing regime for analgesics which is provided in the literature, for example in (“Intrathecal Drug Delivery for the Management of Cancer Pain” A Multidisciplinary Consensus of Best Clinical Practices VOL 3, 6 Nov./Dec. 2005). Fentanyl may be delivered at a rate of 1 mg/day to 2 mg/day, hydromorphone may be delivered at a rate of 5 mg/day, sufentanil may be delivered at a rate of 0.3 mg/day to 1.0 mg/day. Example α2-adrenergic agonists that may be used for treatment of pain include clonidine or clonidine-hyromorpone mixture. Clonidine may be delivered at a rate of 0.01 mg/day to 1 mg/day. When treating pain, it may be desirable to adjust the dose based on the subject's pain. For example, additional analgesic may be delivered if the subject's pain does not decrease or less analgesic may be delivered if the subject's pain decreases. The amount of analgesic delivered may be adjusted by a treating physician or by the patient. Adjustment is made by programming the time interval between dispensing cycles, namely by adjustment of the “off” period. In the dispensing device of the present invention the time interval (“off” period) may be set between 10 to 120 minutes. The time interval may be adjusted until the average deliver rate provides the desired pain relief

The patency of the cochlear aqueduct can be measured by a device that detects the displacement of the tympanic membrane as a result from intracranial pressure variation. CSF pressure changes following posture position transition from supine to sitting. CSF pressure is transmitted via the cochlear aqueduct to perilymph fluid in the inner ear which consequently displaces the tympanic membrane. The rate of the displacement is a function the patency of the cochlear aqueduct. The displacement can be measured by the MMS-10 Tympanic Membrane Displacement Analyzer (Marchbanks Measurements Systems, Lymington Hampshire UK). The patency measurement may be used to assist in selecting the opioid drug to be used for the treatment, the concentration of the drug and its delivery rate. Methods for measuring the patency of the cochlear aqueduct using the MMS-10 was describe in several publications (see e.g., Rosingh et al., Non-invasive perilymphatic pressure measurement in normal hearing subjects using the MMS-10 Tympanic Displacement Analyser, Acta Otolaryngol, 116(3):382 [1996])

Treatment of Eating Disorders

While it will be understood that the method described above are applicable to delivery of small molecules, such as opioid analgesics, some examples of large molecules which have shown therapeutic effectiveness following infusion to CSF may also be delivered in accordance with the methods described.

Animal study (see, Chen G. et al., Preliminary study on brain-targeted drug delivery via inner ear, Yao Xue Xue Bao, 42(10):1102 [2007]) has shown that dexamethason reaches the forth ventricle compartment of the brain following administration via the inner ear. Due to dynamic distribution and bulk flow within the subarachnoid space, CSF that is produced in ventricular system flows from the forth ventricle directly into the cisterna magna. Referring to FIG. 14, a diagrammatic illustration of cerebrospinal fluid flow in the subarachnoid space of a human is shown. The arrows indicate the direction of CSF flow. It can be seen that CSF flow direction 1301 is from the forth ventricle 1302 to the cisterna magna 1303. Cisterna magna 1303 is one of three principal openings in the subarachnoid space which has been used as an access route to the CSF.

U.S. patent application Ser. No. 11/951,778 has shown that delivery of large molecule to the cisterna magna may serve to enhance broad delivery of the therapeutic agent to brain tissue. Using the procedure of cisternal puncture, a catheter can be placed to deliver drugs to the CSF. Delivery via inner ear may used for improved surgical ease relative to cisternal puncture and insertion of a catheter through the scalp to the compartment of the brain.

One therapeutic application that was identified in U.S. patent application Ser. No. 11/951,778 is in administration of peptide hormones and biological active substance to the cisterna magna for modification of eating behavior. Such biological active substance may be a gut hormone, a pancreatic hormone, an adipose tissue derived hormone or a hormone activated by a gut hormone. Modification of eating behavior using the above referenced peptide hormones may be useful in treating or investigating eating disorders, such as obesity, anorexia nervosa, and bulimia. When used for treating a disorder in a human subject, an administered peptide hormone will typically be a recombinant human peptide hormone. Examples of gut hormones that may be used to modify eating behavior include ghrelin, glucagon-like peptide-1, oxyntomodulin, peptide YY, and cholecystokinin. Insulin is an example of a pancreatic hormone that may be useful in modifying eating behavior when delivered to the CSF, while leptin is an example of an adipose tissue derived hormone. Melanocyte-stimulating-hormone (MSH) is a hormone that is activated by a gut hormone, a pancreatic hormone, or an adipose tissue derived hormone that may alter eating behavior.

When treating obesity, it may be desirable to adjust the amount of the polypeptide delivered to the inner ear based on the subject's weight. For example, additional polypeptide may be delivered if the subject's weight does not decrease or less polypeptide may be delivered if the subject's weight decreases. The amount of polypeptide delivered may be adjusted by a treating physician or may be adjusted by a delivery device receiving feedback on the patient's appetitive state. Adjustment is made by programming the time interval that the dispenser operates to dispense polypeptide. It will be understood that overall body weight, body fat percentage or other indicator of obesity may also be used as feedback to adjust the amount of polypeptide delivered to treat obesity. The amount of polypeptide delivered may be varied at certain times of the day, etc. The use of a programmable dispensing device of the present invention may allow for such variations in delivery rate. The peptide hormones described herein may be administered at a suitable dose capable of achieving a desired modification of eating behavior. By way of example, leptin may be administered at a daily dose at a rate of 1×10⁻⁴ mg/kg/day to 1×10⁻¹ mg/kg/day. In various embodiments leptin is administered at a daily dose of between about 1×10⁻³ mg/kg/day to about 1×10⁻² mg/kg/day.

The peptide hormones described herein may be formulated at any suitable concentration to achieve the daily doses according to the selected delivery regimen. By way of example, leptin, such as recombinant leptin, may be formulated at concentrations of between about 10 mg/ml and about 500 mg/ml. In various embodiments leptin is formulated at a concentration of between about 100 mg/ml and about 450 mg/ml.

Peptide hormone compositions include solutions, suspensions, dispersions, and any class of nanocolloidal carrier including polymeric nanoparticles, liposome, polymeric micelles and suspension of solid lipid nanoparticles (SLN). SLN preferably have a median particle size of less than 1 micron and more preferable about 600 nm to maximize drug loading capacity and maintain particles ability to transport via the round-window membrane of the inner ear.

Dispersions may be formulated according to techniques well known in the art (see, for example, Remington's Pharmaceutical Sciences, Chapter 43, 14th Ed., Mack Publishing Co., Easton, Pa.), using suitable dispersing or wetting and suspending agents, such as sterile oils, including synthetic mono- or diglycerides, and fatty acids, including oleic acid. Fluid compositions containing large molecules may be prepared in water, saline, isotonic saline, phosphate-buffered saline, citrate-buffered saline, and the like and may optionally mixed with a nontoxic surfactant. Dispersions may also be prepared in glycerol, liquid polyethylene, glycols, DNA, vegetable oils, triacetin, and the like and mixtures thereof. Under ordinary conditions of storage and use, these preparations may contain a preservative to prevent the growth of microorganisms. Pharmaceutical dosage forms suitable for injection or infusion include sterile, aqueous solutions, suspensions, or dispersions or sterile powders comprising an active ingredient which powders are adapted for the extemporaneous preparation of sterile injectable or infusible solutions or dispersions. Preferably, the ultimate dosage form is a sterile fluid and stable under the conditions of manufacture and storage. A liquid carrier or vehicle of the solution, suspension or dispersion may be a diluent or solvent or liquid dispersion medium comprising, for example, water, ethanol, a polyol such as glycerol, propylene glycol, or liquid polyethylene glycols and the like, vegetable oils, nontoxic glyceryl esters, and suitable mixtures thereof. Proper fluidity of solutions, suspensions or dispersions may be maintained, for example, by the formation of liposomes, by the maintenance of the desired particle size, in the case of dispersion, or by the use of nontoxic surfactants. The prevention of the action of microorganisms can be accomplished by various antibacterial and antifungal agents, for example, parabens, chlorobutanol, phenol, sorbic acid, thimerosal, ethanol and the like. In many cases, it will be desirable to include isotonic agents, for example, sugars, buffers, or sodium chloride. Prolonged absorption of the compositions can be brought about by the inclusion in the composition of agents delaying absorption, for example, aluminum monosterate hydrogels and gelatin. Excipients that increase solubility, such as cyclodextrin, may be added. The concentration of large molecules may be readily determined and varied as conditions warrant based on the disease to be treated or the response of the subject to the treatment. For prolonged delivery of a fluid composition to a subject, it may be desirable for the composition to be isotonic tissue into which the composition is being delivered. For example, the fluid composition may be isotonic with a subject's CSF or perilymphatic fluid. CSF typically has a tonicity of about 305 mOsm. Accordingly, fluid compositions intended for intracochlear delivery may advantageously has a tonicity of about 290 mOsm to about 320 mOsm. If during formulation the composition has a tonicity lower than about 290 mOsm to about 320 mOsm, the tonicity may be enhanced by adding a tonicity enhancing agent, such as sodium chloride. As used herein, “tonicity enhancing agent” means a compound or composition that increases tonicity of a composition. However, such tonicities of between about 290 mOsm to about 320 mOsm are not always achievable. For example, high concentrations of large molecules themselves when dissolved in water may result in a tonicity of greater than 320 mOsm. When the concentration of the large molecule in a fluid composition renders the composition hypertonic relative to a subject's physiological fluid, it is preferred that little or no amount of a tonicity enhancing agent be added to the composition. However, it will be recognized that it may desirable to add one or more additional compounds to the composition even though the addition of the additional compound(s) will further increase tonicity of the composition. For example, it may be desirable to add to the composition an additional therapeutic agent, stabilizing compound, preservative, solubilizing agent, buffer, etc., even though tonicity will be increased. Sterile fluid compositions may be prepared by incorporating the large molecule in the desired amount in the appropriate diluent or solvent with various other ingredients, e.g. as enumerated above, and, as desired, followed by sterilization. Any means for sterilization may be used. For example, sterilization may be accomplished by heating, filtering, aseptic technique, and the like, or a combination thereof. In some circumstances it may be desirable to obtain a sterile powder for the preparation of sterile solutions. Such sterile powders may be prepared by vacuum drying and freeze-drying techniques, which yield a powder of the active ingredient plus any additional desired ingredient present in a previously sterile-filtered solution. Regardless of the large molecule to be delivered, it may be desirable to conjugate the large molecule with a molecule capable of enhancing cellular uptake of the large molecule into cells. Conjugation may be done according to any known or future developed technique with any known or future developed conjugate. One example is the conjugation of polypeptides with mannose, e.g. as described US Patent Publication No. 2005/0208090.

Preferably, the biologically active agent is modified for enhanced cellular uptake to the deep tissue of the brain. The biologically active agent may be a therapeutic protein for treatment of neurological diseases selected from the group consisting of lysosomal storage diseases, protein deficiency diseases, enzyme deficiency diseases, inborn errors of metabolism, neurodegenerative diseases.

The neurological diseases may consisting of Fragile X Syndrome, Parkinson's disease, Alzheimer's disease, and combinations thereof. The therapeutic protein formulation may comprises enzymes providing for enzyme replacement therapy. This includes enzymes selected from the group consisting of beta-glucosidase, glucocerebrosidase, acid sphingomyelinase, galactocerebrosidase, arylsulfatase A, saposin B, alpha-galactosidase A, beta-galactosidase, beta-hexosaminidase A, beta-hexosaminidase A and B, alpha-L-fucosidase, alpha-D-mannosidase, beta-D-mannosidase, N-aspartyl-beta-glucosaminidase, alpha-glucosidase, LAMP-2, glycogen branching enzyme, neuraminidase, phosphotransferase, alpha-L-iduronidase, iduronate-2-sulfatase, heparan-N-sulfatase, alpha-N-acetylglucosaminidase, acetylCoA:N-acetyltransferase, N-acetylglucosamine 6-sulfatase, galactose 6-sulfatase, beta-galactosidase, N-acetylgalactosamine 4-sulfatase, beta-glucuronidase, lysosomal acid lipase, acid cholesteryl ester hydrolase, acid ceramidase, N-acetyl-alpha-D-galactosaminidase, palmitoyl protein thioesterase, and combinations thereof.

The therapeutic protein formulation may also comprises proteins selected from the group consisting of GDNF, FMRP, and combinations thereof.

In the present invention which is configured for intracochlear delivery at least some of the proteins within said therapeutic protein formulation have been modified to comprise a transport aid that provides for enhanced cellular uptake to the tissue of the brain. Proteins may be modified by incorporating into their structure amino acid sequences providing for an intrinsic transport aid. In some cases modification is created by the joining of two or more genes which is also know as fusion proteins.

Proteins may be modified by conjugation to a transport aid that facilitates the cellular uptake of the therapeutic protein itself. Conjugation comprises a linker species which existing between the therapeutic protein and the transport aid. The linker may be selected from the group consisting of peptide linkages, disulfide linkages, and combinations thereof. In some cases the linker may be a streptavidin-biotin complex. Advantageously, the integrity and activity of the protein formulation is achieved by the addition to said therapeutic protein formulation, at least one species operable for maintaining a desired pH.

As used in conjunction with the disclosed invention, the term “biologically active agent” as defined herein, is an agent, or its pharmaceutically acceptable salt, or mixture of compounds, which has therapeutic, prophylactic, pharmacological, physiological or diagnostic effects on a mammal and may also include one compound or mixture of compounds that produce more than one of these effects. Suitable therapeutic, pharmacological, physiological and/or prophylactic biologically active agents can be selected from the following listed, and are given as examples and without limitation: amino acids, anabolics, analgesics and antagonists, anaesthetics, anti-adrenergic agents, antiasthmatics, anti-atherosclerotics, antibacterials, anticholesterolics, anti-coagulants, antidepressants, antidotes, anti-emetics, anti-epileptic drugs including muscimol, antifibrinolytics, anti-inflammatory agents, antihypertensives, antimetabolites, antimigraine agents, antimycotics, antinauseants, antineoplastics, anti-obesity agents, antiParkinson agents, antiprotozoals, antipsychotics, antirheumatics, antiseptics, antivertigo agents, antivirals, appetite stimulants, bacterial vaccines, bioflavonoids, calcium channel blockers, capillary stabilizing agents, coagulants, corticosteroids, detoxifying agents for cytostatic treatment, diagnostic agents (like contrast media, radiopaque agents and radioisotopes), drugs for treatment of chronic alcoholism, electrolytes, enzymes, enzyme 59 inhibitors, ferments, ferment inhibitors, gangliosides and ganglioside derivatives, hemostatics, hormones, hormone antagonists, hypnotics, immunomodulators, immunostimulants, immunosuppressants, minerals, muscle relaxants, neuromodulators, neurotransmitters and nootropics, osmotic diuretics, parasympatholytics, parasympathomimetics, peptides, proteins, psychostimulants, respiratory stimulants, sedatives, serum lipid reducing agents, smooth muscle relaxants, sympatholytics, sympathomimetics, vasodilators, vasoprotectives, vectors for gene therapy, viral vaccines, viruses, vitamins, oligonucleotides and derivatives, and any therapeutic agent capable of affecting.

One skilled in the art will appreciate that the present invention, defining intracochlear drug delivery to the CNS can be practiced with embodiments other than those disclosed. The disclosed embodiments are presented for purposes of illustration and not limitation, and the present invention is limited only by the claims that follow.

What is claimed is:

1. A system for prolonged administration of fluid composition to the perilymphatic fluid of the inner ear, the system comprising:
an implantable otological conduit configured to convey fluid from the auditory ear canal to the round-window membrane, the conduit having one end contacting the round-window membrane and a second end for contacting a fluid source; and
a dispenser for dispensing fluid, wherein the dispenser includes:
a housing holding an electronic module and a cartridge containing a fluid composition to be dispensed; and
a dispensing nozzle connected by a tube to the housing, the dispensing nozzle disposed inside the auditory canal of a subject in close proximity to the otological conduit;
wherein the dispensing nozzle operates intermittently to supply said fluid composition to the otological conduit based on pre-programmed setting controlled by the electronic module.

2. The system of claim 1, wherein the housing is worn behind the ear of the subject.

3. The system of claim 1, wherein the electronic module further comprises a supercapacitor and a charging circuit, and wherein the charging circuit operates to:
charge the supercapacitor using energy from a coin cell battery; and
discharge the supercapacitor through a transducer, thereby supplying power to dispense a quantity of liquid.

4. The system of claim 1, wherein the otological conduit further includes an implantable wick member having a first end and a second end, the first end contacting the round-window membrane of the subject, and the second end extending to the ear canal of the subject.

5. The system of claim 1, wherein the dispenser operates intermittently to dispense fluid to the otological conduit based on a pre-programmed setting, and wherein the period between dispensing cycles is between 10 and 120 minutes.

6. The system of claim 1, wherein the fluid composition further includes nanocolloidal carriers selected from the group consisting of polymeric nanoparticles, liposomes and polymeric micelles.

7. The system of claim 6, wherein the nanocolloidal carriers have a particle size between 50 nm and 1000 nm.

8. A method for treating chronic pain in a subject, the method comprising:
placing a fluid composition comprising one or more analgesics into a cartridge connected to a dispensing nozzle, the analgesics selected from the group consisting of opioid analgesics, α2-adrenergic agonists, and combinations thereof;
implanting an otological conduit in the tympanic membrane of the subject for delivery of the fluid composition from the auditory ear canal to the round-window membrane of the subject;
placing the dispensing nozzle inside the ear canal near the otological conduit; and
dispensing the fluid composition from the dispensing nozzle to the otological conduit at a pre-determined delivery rate such that the fluid composition perfuses through the round-window membrane to the perilymphatic fluid.

9. The method of claim 8, wherein fluid composition comprises the α2-adrenergic agonist clonidine.

10. The method of claim 9, wherein the clonidine is delivered at a rate of between 0.01 mg/day and 1 mg/day.

11. The method of claim 8, wherein the fluid composition comprises one or more opioid analgesics selected from the group consisting of hydromorphone, hydromorphone hydrochloride, morphine sulfate, fentanyl, fentanyl citrate, and sufentanil.

12. The method of claim 11, wherein the fluid composition comprises hydromorphone hydrochloride, and wherein the hydromorphone hydrochloride is delivered at a rate of between 0.5 mg/day and 5 mg/day.

13. The method of claim 11, wherein the fluid composition comprises fentenyl, and wherein the fentenyl is delivered at a rate of between 0.2 mg/day and 2 mg/day.

14. The method of claim 8, wherein the fluid composition further includes nanocolloidal carriers selected from the group consisting of polymeric nanoparticles, liposomes and polymeric micelles.

15. The method of claim 14, wherein the nanocolloidal carriers have a particle size between 50 nm and 1000 nm.

16. A method for treating eating disorders in a subject, the method comprising:
placing a fluid composition comprising peptide hormone into a cartridge connected to a dispensing nozzle, the peptide hormone selected from the group consisting of a gut hormone, an adipose tissue derived hormone, a pancreatic hormone, a hormone activated by a gut hormone, and an adipose tissue derived hormone;
implanting an otological conduit in the tympanic membrane of the subject for delivery of the fluid composition from the auditory ear canal to the round window membrane of the subject;
placing the dispensing nozzle near the otological conduit inside the ear canal; and
dispensing the fluid composition from the cartridge to the otological conduit;
wherein fluid from the cartridge is delivered to the otological conduit at a pre-determined delivery rate such that the fluid composition perfuses through the round-window membrane to the perilymphatic fluid.

17. The method of claim 16, wherein the peptide hormone is selected from the group consisting of ghrelin, glucagon-like peptide-1, oxyntomodulin, peptides, cholecystokinin, leptin, insulin, and melanocyte-stimulating hormone.

18. The method of claim 16, wherein the fluid composition further includes nanocolloidal carriers selected from the group consisting of polymeric nanoparticles, liposomes and polymeric micelles.

19. The method of claim 18, wherein the nanocolloidal carriers have a particle size between 50 nm and 1000 nm.
